(19) United States
(12) Patent Application Publication
(10) Pub. No.: US 2021/0322461 A1
(43) Pub. Date: Oct. 21, 2021

(54) METHOD OF CROSS-LINKING COLLAGEN

(71) Applicant: Alpha Phase Technologies LLC, Athens, OH (US)

(72) Inventor: David BURNETTE, Lancaster, OH (US)

(21) Appl. No.: 16/849,138

(22) Filed: Apr. 15, 2020

(51) Int. Cl. A61K 33/00; C08J 3/24; A61P 27/02

(52) U.S. Cl. CPC A61K 33/00; C08J 2389/00; A61P 27/02; C08J 3/24

(57) ABSTRACT

A method of cross-linking collagen comprises generating reactive species from a non-thermal plasma and delivering the reactive species to a collagen-containing target to induce collagen cross-linking. In a specific embodiment, the reactive species can be a reactive oxygen species, for example singlet oxygen. A method of cross-linking collagen comprises generating reactive oxygen species from a non-thermal plasma and delivering the reactive species to a collagen-containing target to induce collagen cross-linking, wherein the non-thermal plasma is generated from a non-flammable gas medium comprising an inert buffer gas, the reactive oxygen species is selected from the group consisting of a superoxide ion radical, hydroxyl radical, peroxide, hydrogen peroxide, organic peroxide, or a combination thereof, and the collagen-containing target is corneal tissue and wherein the collagen cross-linking induces cross-linking within and between collagen fibers in the corneal tissue.

### FIELD OF THE INVENTION

The present invention relates to methods of cross-linking collagen comprising generating reactive species from a non-thermal plasma and delivering the reactive species to a collagen-containing target to induce collagen cross-linking. The present invention also relates to methods of cross-linking collagen comprising generating reactive oxygen species from a non-thermal plasma and delivering the reactive species to a collagen-containing target to induce collagen cross-linking, wherein the non-thermal plasma is generated from a non-flammable gas medium comprising an inert buffer gas, the reactive oxygen species is selected from the group consisting of a superoxide ion radical, hydroxyl radical, peroxide, hydrogen peroxide, organic peroxide, or a combination thereof, and the collagen-containing target is corneal tissue and wherein the collagen cross-linking induces cross-linking within and between collagen fibers in the corneal tissue.

### BACKGROUND OF THE INVENTION

Corneal ectatic diseases encompass a number of non-inflammatory eye conditions and are generally characterized by the thinning of the central, paracentral, or peripheral cornea. One of the most common corneal ectatic diseases, keratoconus, is a progressive eye disease that is characterized by bulging of the cornea, wherein the cornea takes on a cone-like shape. As a result of this cone-like shape, the cornea begins to deflect light as it enters the eye, thereby leading to distorted vision.

Patients with keratoconus experience a variety of symptoms, including, inter alia, blurred or distorted vision, vision loss, astigmatism, double vision, nearsightedness, increased sensitivity to light and/or glare, a sudden worsening or clouding of vision, and/or frequent changes in eyeglass prescriptions. While the exact cause of keratoconus is unknown, risk factors are believed to include having a family history of keratoconus, vigorous eye rubbing, or having conditions such as retinitis pigmentosa, Down syndrome, Ehlers-Danlos syndrome, hay fever and/or asthma.

Treatment of keratoconus is evaluated on a case-by-case basis and depends on the severity of the condition. In the early stages of keratoconus, treatment options include, for example, eyeglasses, soft contact lenses, or corneal inserts; however, soft contact lenses may need to be replaced with rigid, gas permeable lenses as the condition progresses. In cases of advanced keratoconus, the cornea may become scarred, thus severely impacting the patient's vision. In these later stages of the disease, treatment may go so far as to require corneal transplantation. However, before resorting to surgery, an additional treatment option can be collagen cross-linking.

Corneal collagen cross-linking (CXL) is a minimally invasive FDA-approved process for strengthening the mechanical properties of the cornea in patients suffering from keratoconus or other forms of corneal ectasia. During the procedure, the cornea is saturated with riboflavin and exposed to an ultraviolet LED. Unfortunately, conventional CXL methods require that a patient's eye be exposed directly to ultraviolet LED for a period of time. The effectiveness of existing CXL methods is thus limited by the maximum UV energy that can be applied without cytotoxic effects. It would therefore be advantageous to provide an improved method of CXL that does not require the use of ultraviolet LED light.

In conventional methods of CXL, the wavelength of ultraviolet LED is chosen to coincide with an absorption peak of riboflavin. The absorption of incident photons, shown in Equation (1), causes electronic excitation of riboflavin to the singlet state, which freely radiates back to the ground state or to the lower-lying triplet state, as shown in Equation (2). In the equations below, hυ corresponds to the energy of the absorbed or emitted photons. Rf is an abbreviation for riboflavin. 1Rf*, 3Rf*, identify excited states, wherein the number, i.e., 1 or 3, relates to the quantum spin number. By way of example, Rf can be called the ground state, and 1Rf can be called the excited state after absorption. Kamaev, P., Friedman, M. D., Sherr, E., & Muller, D. (2012). Photochemical Kinetics of Corneal Cross-Linking with Riboflavin. Investigative Ophthalmology & Visual Science, 53(4); 2360-2367.

Rf+hυ_{ab}→¹Rf* (1)

¹Rf*→³Rf*+hυₑₘ (2)

The mechanisms at work in CXL are quite complex and not fully understood. However, it is commonly believed that the triplet state of riboflavin is free to react directly with neighboring collagen fibers or with dissolved oxygen. In the latter process, the excited riboflavin is quenched to form reactive oxygen species (ROS), including singlet oxygen (¹O₂), an electronically-excited variant of atmospheric oxygen. Turkcu, U. O., Yuksel, N., Novruzlu, S., Yalinbas, D., Bilgihan, A., & Bilgihan, K. (2016). Protein Oxidation Levels After Different Corneal Collagen Cross-Linking Methods. Cornea, 35(3), 388-391. The relative importance of ¹O₂ versus other ROS, such as superoxide (O₂⁻), the hydroxyl radical (OH), or hydrogen peroxide (H₂O₂), is not entirely known, although the use of deuterium oxide drops, commonly known to extend the lifetime of ¹O₂, are used in conventional CXL. It is also noteworthy that the lifetimes of various ROS are very dissimilar and the CXL process is known to be non-uniform with depth into the cornea.

More importantly, it has been well-established that the effectiveness of CXL is limited by the rate of oxygen diffusion into the cornea. Because of this, variants of CXL using a pulsed light source have emerged in research. In this approach, the LED is activated when enough time has passed for oxygen to have diffused into the stroma. It is widely accepted that the photochemical reaction using riboflavin leads to the generation of ROS, which subsequently enables cross-linking. It is also well known that this process is diffusion limited. This is most evident based on studies showing that the process shows superior results when the epithelial cells, a barrier, are removed from the surface of the cornea. See Turkcu et al., 2016.

As indicated above, available comeal cross-linking treatment requires direct exposure of a patient's eye to ultraviolet LED light , which has drawbacks, for example the effectiveness of the process is limited by the maximum UV energy that can be applied without cytotoxic effects. Exposing the eye to ultraviolet light can lead to damage, some of which may be irreversible, on both the endothelial layer and exterior of the eye. Accordingly, improved methods of collagen cross-linking, and more specifically corneal cross-linking, are desirable.

### SUMMARY OF THE INVENTION

According to the present invention, there is provided a method of cross-linking collagen, comprising generating reactive species from a non-thermal plasma and delivering the reactive species to a collagen-containing target to induce collagen cross-linking.

In another embodiment, there is provided a method of cross-linking collagen, comprising generating reactive oxygen species from a non-thermal plasma and delivering the reactive species to a collagen-containing target to induce collagen cross-linking, wherein the non-thermal plasma is generated from a non-flammable gas medium comprising an inert buffer gas, the reactive oxygen species is selected from the group consisting of a superoxide ion radical, hydroxyl radical, peroxide, hydrogen peroxide, organic peroxide, or a combination thereof, and the collagen-containing target is corneal tissue and wherein the collagen cross-linking induces cross-linking within and between collagen fibers in the corneal tissue.

The methods of cross-linking collagen according to the present invention are advantageous in that, when administered in combination with existing methods of CXL, they are able to reduce the amount of ultraviolet LED light (UV light) that a patient is exposed to. In addition, the methods of the present invention are also advantageous in that, when used as the sole method of CXL, they are able to eliminate use of UV light completely. This and additional objects and advantages of the invention will be more fully apparent in view of the following detailed description.

### BRIEF DESCRIPTION OF THE DRAWINGS

The embodiments set forth in the drawings are illustrative of certain aspects of the invention and exemplary in nature and are not intended to limit the invention defined by the claims, wherein:

FIG. 1 illustrates an embodiment of the present invention, wherein a reactive species is generated from a non-thermal plasma and delivered to corneal tissue to induce cross-linking within and between collagen fibers in the cornea, as described in Example 1.

### DETAILED DESCRIPTION

Specific embodiments of the invention are described herein. The invention can, however, be embodied in different forms and should not be construed as limited to the embodiments set forth herein. Rather, these embodiments are provided to illustrate more specific features of certain aspects of the invention to those skilled in the art.

The terminology as set forth herein is for description of the embodiments only and should not be construed as limiting the disclosure as a whole. All references to singular characteristics or limitations of the present disclosure shall include the corresponding plural characteristic or limitation, and vice versa, unless otherwise specified or clearly implied to the contrary by the context in which the reference is made. Unless otherwise specified, “a,” “an,” “the,” and “at least one” are used interchangeably. Furthermore, as used in the description and the appended claims, the singular forms “a,” “an,” and “the” are inclusive of their plural forms, unless the context clearly indicates otherwise.

To the extent that the term “includes” or “including” is used in the description or the claims, it is intended to be inclusive of additional elements or steps, in a manner similar to the term “comprising” as that term is interpreted when employed as a transitional word in a claim. Furthermore, to the extent that the term “or” is employed (e.g., A or B), it is intended to mean “A or B or both.” When the “only A or B but not both” is intended, then the term “only A or B but not both” is employed. Thus, use of the term “or” herein is the inclusive, and not the exclusive use. When the term “and” as well as “or” are used together, as in “A and/or B” this indicates A or B as well as A and B.

The methods described in the present disclosure can comprise, consist of, or consist essentially of any of the elements and steps as described herein.

All ranges and parameters, including but not limited to percentages, parts, and ratios disclosed herein are understood to encompass any and all sub-ranges subsumed therein, and every number between the endpoints. For example, a stated range of “1 to 10” should be considered to include any and all sub-ranges beginning with a minimum value of 1 or more and ending with a maximum value of 10 or less (e.g., 1 to 6.1, or 2.3 to 9.4), and to each integer (1, 2, 3, 4, 5, 6, 7, 8, 9, and 10) contained within the range.

Any combination of method or process steps as used herein can be performed in any order, unless otherwise specified or clearly implied to the contrary by the context in which the referenced combination is made.

All percentages are percentages by weight unless otherwise indicated.

The term “inert buffer gas” as used herein, unless otherwise specified, refers to an inert gas that adds pressure to a system. The buffer gas should hold and transfer energy, but should not chemically react itself.

The term “non-flammable gas medium” as used herein, unless otherwise specified, refers to the non-flammable gas that moves through a plasma reactor and undergoes electrical breakdown. The non-flammable gas serves as a source for generating reactive species.

The term “non-thermal plasma” as used herein, unless otherwise specified, refers to a partly ionized gas that is not in thermodynamic equilibrium. Non-thermal plasma (NTP) thus encompasses cold plasma or non-equilibrium plasma.

The term “plasma exhaust gas” as used herein, unless otherwise specified, refers to gas that no longer possesses significant ionization. Plasma exhaust gas thus encompasses gas that is removed from the system, for example from an area around a collagen-containing target.

The term “reactive species” as used herein, unless otherwise specified, refers to a reactive chemical species containing atoms, molecules, radicals, electronically-excited molecules, excited variants of atoms, and/or atoms or molecules possessing significant internal energy to overcome the activation barrier of a reaction at low temperature. Examples of reactive species may include chemically reactive species containing nitrogen, chemically reactive species containing oxygen, and/or excited variants of inert atoms, such as metastable argon and helium. The reactive species that are generated will depend on the non-flammable gas medium that is used.

Plasma, commonly referred to as the fourth state of matter, possesses a large degree of ionization, which is normally associated with extremely high temperatures when a system is in thermal equilibrium. However, NTPs are often generated at biologically compatible temperatures. In most man-made NTPs, free electrons are accelerated by an applied electric field and are characterized by temperatures far in excess of the surrounding gas. Inelastic collisions between these electrons and neutral molecules leads to ionization, dissociation, and electronic excitation, among other reactions. Equation 3 shows the creation of ¹O₂ by direct electron impact as an example. However, this state can also be created in other processes with plasma reactive species. The reactive species generated depends predominantly on the gas composition and a property known as the reduced electric field (E/n), which is related to the electron energy distribution function or electron temperature. Species generated within the plasma range from very short-lived, radiative states to metastable ones.

O₂+e→¹O*₂+e (3)

The kinetics of NTPs are very complex and ROS generation, for example, can require a large number of reactions and special software tools to properly predict. While ROS can be generated directly in the plasma, others may be indirectly created when the plasma exhaust is expelled into an atmosphere. For example, a metastable electronically-excited argon atom (Ar*) holds over 11.5 eV of potential energy, more than enough to dissociate or cause the electronic excitation of atmospheric molecules, including water. In the case of water, the products could be both short-lived (OH) or long-lived (H₂O₂) species.

There has been extensive growth in the research of potential medical applications for these systems. For example, NTPs have been shown to selectively target cancer cells, possibly a combination of both the oxidative stress from the resulting ROS as well as the enhanced permeability resulting from the pulsed electric fields. Due to advances in plasma kinetic modeling and laser diagnostics, the complex nature of plasma chemistry has seen extensive study in the last decade. It is now possible to adjust gas composition, as well as electrode configuration and the applied voltage pulse, to generate a desired payload of ROS at essentially room temperature.

The present invention thus provides an improved method of collagen cross-linking comprising generating a reactive species 1 from a non-thermal plasma 3 and delivering the reactive species 1 to a collagen-containing target to induce collagen cross-linking. Without wishing to be bound by any particular theory, the methods of the present invention are based on the principle that the photochemical process involved in conventional CXL can be enhanced by limiting the amount of UV exposure and/or replaced by directing a NTP or its afterglow/effluent onto the target, for example the cornea, wherein the generated ROS will encourage collagen cross-linking. Projecting the NTP onto the surface of the cornea is not required for the creation of ROS and is likely undesirable in live patients. While the distance from the cornea to the plasma source will vary, it should be a distance that will allow the charged particles to recombine by the time the gas reaches the cornea. In a specific embodiment, the ideal distance from the cornea to the NTP source will be in the range of about 1 mm to about 1 m.

In further embodiments, the distance from the cornea to the plasma source can be about 1 mm to about 10 mm, about 1 mm to about 20 mm, about 1 mm to about 30 mm, about 1 mm to about 40 mm, about 1 mm to about 50 mm, about 1 mm to about 60 mm, about 1 mm to about 70 mm, about 1 mm to about 80 mm, about 1 mm to about 90 mm, about 1 mm to about 100 mm, about 1 mm to about 200 mm, about 1 mm to about 300 mm, about 1 mm to about 400 mm, about 1 mm to about 500 mm, about 1 mm to about 600 mm, about 1 mm to about 700 mm, about 1 mm to about 800 mm, about 1 mm to about 900 mm, about 100 mm to about 200 mm, about 100 mm to about 300 mm, about 100 mm to about 400 mm, about 100 mm to about 500 mm, about 100 mm to about 600 mm, about 100 mm to about 700 mm, about 100 mm to about 800 mm, about 100 mm to about 900 mm, about 200 mm to about 300 mm, about 200 mm to about 400 mm, about 200 mm to about 500 mm, about 200 mm to about 600 mm, about 200 mm to about 700 mm, about 200 mm to about 800 mm, about 200 mm to about 900 mm, about 300 mm to about 400 mm, about 300 mm to about 500 mm, about 300 mm to about 600 mm, about 300 mm to about 700 mm, about 300 mm to about 800 mm, about 300 mm to about 900 mm, about 400 mm to about 500 mm, about 400 mm to about 600 mm, about 400 mm to about 700 mm, about 400 mm to about 800 mm, about 400 mm to about 900 mm, about 500 mm to about 600 mm, about 500 mm to about 700 mm, about 500 mm to about 800 mm, about 500 mm to about 900 mm, about 600 mm to about 700 mm, about 600 mm to about 800 mm, about 600 mm to about 900 mm, about 700 mm to about 800 mm, about 700 mm to about 900 mm, about 800 mm to about 900 mm, or about 900 mm to about 1 m.

In a specific embodiment, the non-thermal plasma 3 is generated from a non-flammable gas medium 7. As previously mentioned, the reactive species 1 that are generated will depend on the non-flammable gas medium 7 that is used. In a specific embodiment, the reactive species 1 is a reactive oxygen species. The reactive oxygen species is preferably selected from a superoxide ion radical, hydroxyl radical, peroxide, hydrogen peroxide, organic peroxide, or a combination thereof. In a specific embodiment, the reactive oxygen species is singlet oxygen.

The method of the invention can be used to deliver the reactive species 1 to a collagen-containing target to induce collagen cross-linking, for example in applications such as tissue engineering or bone grafting. However, in a specific embodiment, the collagen-containing target is a collagen-containing tissue. In a specific embodiment, the tissue is corneal tissue 5. In a specific embodiment, the collagen cross-linking induces cross-links within and between collagen fibers in the cornea, thereby stabilizing the cornea.

As indicated above, corneal transplantation is one of the treatment options for a patient with a more severe case of keratoconus. Unfortunately, there are several risks associated corneal transplantation, including, for example, cornea graft rejection, eye infection, and/or problems with the use of stitches. Cornea graft rejection is the one of the most serious complications after a corneal transplant. Thus, in a specific embodiment of the invention, the cross-linking provides new links between collagen fibers within the cornea following a corneal transplant.

In a further embodiment of the invention, the reactive species are delivered to a collagen-containing target in an ocular region of a subject suffering from an ocular disease. In a specific embodiment, the ocular disease is a corneal ectatic disease. While the method of the present invention can be used in a variety of treatments in which strengthening of the cornea would be advantageous, the corneal ectatic disease is preferably selected from keratoconus, pellucid marginal degeneration, keratoglobus, postkeratorefractive ectasia, wound ectasia after penetrating keratoplasty (PK), and/or ectasia after receiving laser-assisted in-situ keratomileusis (LASIK) eye treatment.

With regard to NTPs, there are many subcategories of NTP and the descriptions of these systems change with the particular configuration and power supply selected. Spark gaps, corona, fast ionization waves, plasma jets, gliding arcs, and glow discharges are all terms for NTP generated by an applied electric field. These can be created with direct current (DC) or pulsed DC power, AC power, microwave cavities, nanosecond pulses, and so on. There are also laser-produced and inductively coupled (magnetically driven) plasmas. In fact, there are many combinations of electrode configuration and applied waveforms that can be used to generate the mentioned reactive flow, however the RF-driven DBD is the most likely configuration that can be readily adapted for clinical use. More complex electrode configurations or hybrid (nanosecond +DC, etc.) power supplies require further research to confirm that they do not interfere with other clinical equipment, but may ultimately prove more efficient. Regardless of the arrangement, a plasma source will yield a vast reactive species payload.

Thus, in accordance with a specific embodiment of the invention, the non-thermal plasma is generated using an applied electric field or an applied magnetic field. In a further specific embodiment, the non-thermal plasma is generated using a power source 15 selected from pulsating direct current (PDC), radio frequency (RF), microwave cavities, a nanosecond pulse generator, an induction coil, alternating current dielectric barrier discharge (AC DBD), or combinations thereof. In a specific embodiment, the power source is RF. There are several benefits with using RF as the power source. RF power supplies have been in use for a long time and RF plasma properties are well known. RF power supplies do not put out as much electromagnetic interference (EMI) as, for example, nanosecond pulses. Further, they can be easily load matched, as opposed to a nanosecond power supply wherein a large fraction of the applied power is reflected and dissipated as heat within the supply. Additionally, the frequency of RF plasmas is high enough to create a uniform plasma. All of these advantages make RF a preferred power source to be used in the methods of the present invention.

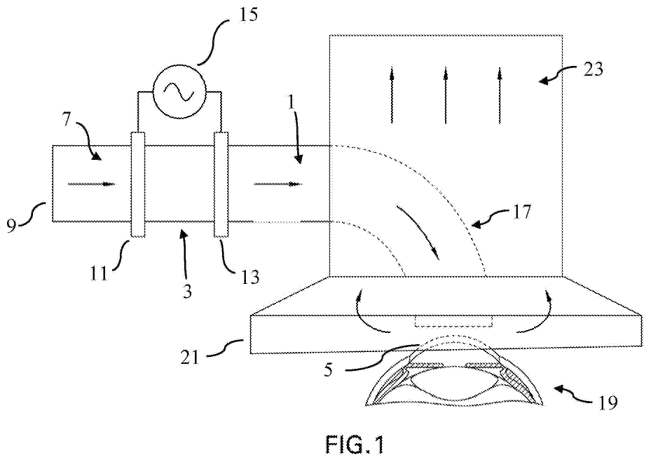

Regarding the non-flammable gas medium 7 of the NTP 3, the method of the invention can be carried out using a variety of gases, including air, but ROS yield will prove larger when nitrogen chemistry is removed. This entails using oxygen in an inert gas such as helium or argon, as opposed to air. By way of example, FIG. 1 of Park, G., Lee, H., Kim, G., & Lee, J. K. (2008). Global Model of He/O₂ and Ar/O₂ Atmospheric Pressure Glow Discharges. Plasma Processes and Polymers, 5(6), 569-576, incorporated herein by reference, shows selected reactive species calculations for helium (a) and argon (b) flows containing only 1% oxygen. The specific conditions of this study include an RF power supply (13.56 MHz) with a power of 100 W/cm³, 1 atm pressure, and 1% O₂ in either helium or argon. Despite this small fraction, 90% of the energy deposited by electrons was deposited into oxygen.

A non-flammable gas medium 7, for example an inert gas such as helium or argon, will not quench metastable oxygen, but will still act as an energy carrier to generate ROS at the surface of the cornea. Thus, in a specific embodiment of the invention, the non-flammable gas medium 7 comprises an inert buffer gas. In a specific embodiment of the invention, the inert buffer gas is selected from helium and argon.

In additional embodiments, the method of the invention further comprises delivering deuterium oxide in aqueous solution to the collagen-containing target before or during delivery of the reactive oxygen species to the selected region of the target. In a specific embodiment, the aqueous solution of deuterium oxide is a hyperoxygenated solution. The dissolved oxygen in deuterium oxide is a function of the partial pressure of the oxygen in the gas around it. Providing the aqueous solution of deuterium oxide as a hyperoxygenated solution, for example by storing the deuterium oxide in an oxygenated solution, or pressurizing it, serves to increase the O₂ content in the solution that is added to the cornea and thus increase the rate of O₂ diffusion into the cornea.

In a further embodiment of the invention, the method further comprises removing plasma exhaust gases 23 from an area around the collagen-containing target. The purpose of doing so is to limit the diffusion of nitrogen into the collagen-containing target, for example collagen-containing tissue such as corneal tissue 5. With regard corneal tissue 5 specifically, this also allows for control of the reactions that are taking place near the eye 19. This can be done, for example, through the use of an exhaust fan connected to a suitable cover over the eye.

In another specific embodiment, the method further comprises concurrently delivering light from a pulsed light-emitting diode (LED) to the collagen-containing target. While the present invention is designed to replace conventional CXL methods in view of its ability to completely eliminate the need to use UV light, it is still possible to use the methods of the invention in combination with existing CXL methods. As mentioned above, the methods of the present invention can enhance existing methods of CXL by reducing the amount of UV light needed to achieve adequate diffusion of O₂ into the cornea.

### EXAMPLE

### Example 1: Delivery of Reactive Species to Corneal Tissue

This example describes a specific embodiment of the present invention, as illustrated in FIG. 1. A reactive species 1 is generated from a non-thermal plasma 3 and is delivered to corneal tissue 5 in order to induce cross-linking within and between collagen fibers in the corneal tissue 5. In this particular configuration, a non-flammable gas medium 7 made up of oxygen and an inert buffer gas, for example diluted 02 in a balance of argon gas, passes through a non-conductive tube 9, such as a quartz tube. Two ring electrodes 11, 13 are provided on the outside of the tube 9 and are connected to an RF power source 15, such as a 13.56 MHz power supply, which causes electrical breakdown of the portion of the argon gas flow 7 between them. The non-thermal plasma 3 may be extended outside of the area defined by these electrodes 11, 13 with an appropriate change in power, frequency, and/or gas speed. The reactive species 1 mixture exiting the non-thermal plasma 3 then passes through a nozzle 17 to be directed onto the exposed corneal tissue 5. The ambient environment around the eye 19 may be controlled via a shroud 21 that that covers the patient's eye 18 and moves exhaust gases 23 away from the treatment area to be properly vented.

What is claimed is:

1. A method of cross-linking collagen, comprising:
generating reactive species from a non-thermal plasma; and
delivering the reactive species to a collagen-containing target to induce collagen cross-linking.

2. The method of claim 1, wherein the non-thermal plasma is generated from a non-flammable gas medium.

3. The method of claim 1, wherein the reactive species are a reactive oxygen species.

4. The method of claim 3, wherein the reactive oxygen species is selected from the group consisting of a superoxide ion radical, hydroxyl radical, peroxide, hydrogen peroxide, organic peroxide, or a combination thereof.

5. The method of claim 4, wherein the reactive oxygen species comprises singlet oxygen.

6. The method of claim 1, wherein the collagen-containing target is a collagen-containing tissue.

7. The method of claim 6, wherein the collagen-containing tissue is corneal tissue.

8. The method of claim 7, wherein the collagen cross-linking induces cross-linking within and between collagen fibers in the corneal tissue.

9. The method of claim 8, wherein the cross-linking provides new crosslinks between collagen fibers within the cornea following a corneal transplant.

10. The method of any one of claim 1, wherein the reactive species are delivered to a collagen-containing target in an ocular region of a subject suffering from an ocular disease.

11. The method of claim 10, wherein the ocular disease is a corneal ectatic disease.

12. The method of claim 11, wherein the corneal ectatic disease is selected from the group consisting of keratoconus, pellucid marginal degeneration, keratoglobus, postkeratorefractive ectasia, wound ectasia after penetrating keratoplasty (PK), ectasia after receiving laser-assisted in-situ keratomileusis (LASIK), and combinations of two or more thereof.

13. The method of claim 1, wherein the non-thermal plasma is generated using an applied electromagnetic field, applied electric field, or an applied magnetic field.

14. The method of claim 13, wherein the non-thermal plasma is generated using a power source selected from the group consisting of pulsating direct current (PDC), radiofrequency (RE), microwave cavities, a nanosecond pulse generator, an induction coil, alternating current dielectric barrier discharge (AC DOD), or combinations thereof.

15. The method of claim 14, wherein the power source is radiofrequency (RF).

16. The method of claim 2, wherein the non-flammable gas medium comprises an inert buffer gas.

17. The method of claim 16, wherein the inert buffer gas comprises helium or argon.

18. The method of claim 1, wherein the method further comprises delivering deuterium oxide in aqueous solution to the collagen-containing target before or during delivery of the reactive species to the collagen-containing target.

19. The method of claim 18, wherein the aqueous solution of deuterium oxide is a hyperoxygenated solution.

20. The method of claim 1, wherein the method further comprises removing plasma exhaust gases from an area around the collagen-containing target.

21. The method of claim 1, wherein the distance from the non-thermal plasma to the collagen-containing target is about 1 mm to about 1 m.

22. The method of claim 1, wherein the method further comprises concurrently delivering light from a pulsed light-emitting diode (LED) to the collagen-containing target.

23. A method of cross-linking collagen, comprising:
generating reactive oxygen species from a non-thermal plasma; and
delivering the reactive species to a collagen-containing target to induce collagen cross-linking;
wherein the non-thermal plasma is generated from a non-flammable gas medium comprising an inert buffer gas;
the reactive oxygen species is selected from the group consisting of a superoxide ion radical, hydroxyl radical, peroxide, hydrogen peroxide, organic peroxide, or a combination thereof; and
the collagen-containing target is corneal tissue and wherein the collagen cross-linking induces cross-linking within and between collagen fibers in the corneal tissue.
